(19) United States
(12) Patent Application Publication
(10) Pub. No.: US 2011/0301553 A1
(43) Pub. Date: Dec. 8, 2011

(54) Antimicrobial lubricant

(75) Inventors: David Goral, Brookfield, CT (US); Harsh Chheda, Cheshire, CT (US); Jane Wronka, Wallingford, CT (US)

(73) Assignee: Smiths Medical ASD, Inc., Rockland, MA (US)

(21) Appl. No.: 12/801,348

(22) Filed: Jun. 4, 2010

(51) Int. Cl. A61L 15/46; A01N 31/14; B05D 1/02

(52) U.S. Cl. 604/265; 427/2.3; 514/721

(57) ABSTRACT

An antimicrobial lubricant combines an antimicrobial agent, preferably silver based, with a silicone lubricant. This is achieved by first formulating a lubricant solution that has a non-volatile silicone lubricant and a volatile solvent carrier. An antimicrobial agent is added to the lubricant solution. The solution is then subjected to ultrasonic energy so that the antimicrobial agent is uniformly mixed with the lubricant in the solution. The solution containing the antimicrobial lubricant may be sprayed onto a medical device, for example the outer surface of the catheter of a peripheral IV catheter. After the solvent flashes off, a coat of uniform antimicrobial lubricant is formed on the catheter surface. The antimicrobial lubricant coating facilitates the insertion of the catheter to a patient, and at the same time prevents the growth of microorganisms from the device and infection to the patient.

### FIELD OF THE INVENTION

The instant invention relates to an antimicrobial lubricant and preferably a silver based antimicrobial agent combined with a silicone based lubricant to form an antimicrobial lubricant adapted to be used on a medical device.

### BACKGROUND OF THE INVENTION

There is disclosed in the prior art antimicrobial agents combined with lubrication agents. Li et al. U.S. Pat. No. 6,214,777 discloses a lubricant composition that uses a quaternary phosphonium compound as an antimicrobial agent combined with a lubricating agent to lubricate containers and/or conveyor systems for containers or receptacles that are used to hold food or beverages in the food or beverage industry. The lubricating agents disclosed include water, amines such as fatty amines, fatty acids, sarcosinates, phosphate esters, water-soluble or water dispersible homopolymers or copolymers of (alkoxy)alkylene glycols, alcohol ethoxycarboxylates such as Neodox available from Hickson Danchem, and water soluble or water dispersible oils.

DeLeon et al. U.S. Pat. No. 4,952,419 discloses the coating of the surface of an implant device with a silicone oil, and the application of an antimicrobial agent in the form of a film adherent powder or dust to the silicone film on the surface of the implant, so as to inhibit infection from the implant.

Khan et al. U.S. Pat. No. 4,925,668 discloses a hydrophillic polymeric medical article that is coated with a combination of chlorhexidine and a silicone lubricant on its surface. The coating is applied by dipping the surface of the article into a solvent solution that contains the anti-infective agent and the lubricant. The layer of chlorhexidine salt and silicone lubricant forms onto the surface of the high melting hydrophillic polymer device after the evaporation of the solvent.

The above combinations of antimicrobial agents and lubrication agents do not deal with medical devices such as peripheral IV catheters, or other similar devices that have catheters adapted to be inserted to a patient. Currently, the inventor is not aware of any peripheral IV catheters with antimicrobial properties on the market.

### SUMMARY OF THE PRESENT INVENTION

The instant invention relates to an antimicrobial lubricant that has an antimicrobial agent, preferably silver based, in combination with a silicone lubricant applied to a medical device such as a peripherical intravenous (IV) catheter. Most antimicrobial technologies in medical devices currently on the market either incorporate the antimicrobial agent into the bulk of the polymer/catheter itself or apply an adherent coating on the surface of the catheter. The antimicrobial lubricant of the instant invention, in contrast, may be coated onto the peripheral IV catheters and other medical devices by being sprayed thereon from a silicone lubricant solution having the antimicrobial agent mixed therein.

By spraying the antimicrobial lubricant onto the surface of the medical device, no additional processing step is required since the base polymer of the medical device, for example the catheter of the IV catheter, is not modified. And the process is straightforward in that the antimicrobial lubricant, in the form of a mist, is evenly sprayed onto the medical device. In the case of a peripheral IV catheter where a needle (or trocar) and the catheter (or cannula) are movable relative to each other, the antimicrobial lubricant may be coated onto the outer diameter surface of the catheter that comes into contact with the patient. The coating on the outer diameter surface of the catheter makes the outer diameter surface of the catheter slippery and smooth and therefore eases or facilitates the insertion of the catheter into the patient. At the same time, the antimicrobial property of the antimicrobial lubricant coating prevents or substantially eliminates the growth of micro-organisms at the patient site and the area adjacent thereto where the medical device comes into contact with the patient. In the case of a peripheral IV catheter and other medical devices that have a catheter or needle that makes contact interaction with a patient, the antimicrobial agent also serves to reduce the catheter and/or needle related bloodstream infections.

Either the outer diameter surface of the needle that comes into contact with the inner diameter surface of the catheter, or the inner diameter surface of the catheter that comes into contact with the outer diameter surface of the needle, or both of the contacting surfaces of the catheter and needle, may also be coated with the antimicrobial lubricant to facilitate the relative movement between the catheter and the needle and to prevent microorganism growth at the interface between the needle and the catheter.

The preferred antimicrobial agent for the instant invention is silver based, such as for example silver particles, carrier based ionic silver and other silver compounds. Two of the preferred silver based antimicrobial agents are Ag-G (glass carrier based silver antimicrobial) and Ag-Z (zeolite carrier based silver antimicrobial). A non-silver based antimicrobial that may also be used is Triclosan. The preferred silicone lubricant is polydialkylsiloxanes, preferably polydimethyl siloxane.

The inventive antimicrobial lubricant solution is formed from a solution containing a silicone oil lubricant in a solvent carrier, with the antimicrobial agent mixed in the solution. The solution is sprayed onto a medical device, for example the outer diameter surface of the catheter of a peripheral IV catheter, and possibly also the base of the catheter device wherefrom the catheter extends. After the solvent carrier flashes off, a layer or film of the antimicrobial lubricant is left on the outside diameter surface of the catheter. Another coat of the antimicrobial lubricant may be added to ensure that the entire outside diameter surface of the catheter is covered by the antimicrobial lubricant, so that the catheter can readily be inserted to the patient due to its slippery and smooth surface.

The present invention is therefore directed to a medical device comprising a lubricious silver based antimicrobial coating that acts as a lubricant to facilitate the contact interaction of the device with a patient and a deterrent to prevent or substantially eliminate the growth of microorganisms on the device and at least those portions of the patient that the device comes into contact with to thereby prevent infection to the patient.

The present invention is also directed to an antimicrobial lubricant that has a silver based antimicrobial agent combined with a silicone based lubricant, the antimicrobial lubricant adapted to coat a medical device for use with a patient, the antimicrobial lubricant acts as a lubricant to facilitate the contact interaction of the device with the patient, the antimicrobial lubricant further acting as a deterrent to prevent or substantially eliminate the growth of microorganisms on the device and at least those portions of the patient that the device comes into contact with to thereby prevent infection to the patient.

The present invention is further directed to a catheter device comprising a needle having an outer diameter extending through and movable relative to a catheter having an inner diameter, the outer diameter surface of the needle in contact with the inner diameter surface of the catheter, the catheter having coated along its length at its outer diameter surface a lubricious antimicrobial coating that facilitates the insertion of the catheter and the needle to a patient and acts as a deterrent to prevent or substantially eliminate the growth of microorganisms at the device and at least those portions of the patient that the device comes into contact with to thereby prevent infection to the patient.

The present invention is furthermore directed to a non-silver based antimicrobial lubricant comprising an antimicrobial agent mixed with a silicone based lubricant in a solution, wherein the antimicrobial agent is Triclosan and the lubricant includes polydialkylsiloxanes preferably polydimethyl siloxane, the solution including a volatile solvent, wherein the Triclosan is mixed with the polydimethyl siloxane in the solution, the solution applied to the surface of a medical by spraying, whereby after the volatile solvent evaporates, the surface of the medical device is coated by a layer of the antimicrobial lubricant.

The present invention is moreover directed to a method of making an antimicrobial lubricant comprising the steps of: a) combining a solvent and a silicone base lubricant to formulate a lubricant solution; b) adding a silver based antimicrobial agent in the lubricant solution; c) subjecting the antimicrobial agent added lubricant solution to ultrasonic energy to mix the antimicrobial agent with the silicone based lubricant to form an antimicrobial lubricant solution; and d) evaporating the solvent from the antimicrobial lubricant solution to obtain the antimicrobial lubricant.

### BRIEF DESCRIPTION OF THE FIGURES

FIG. 1 is a perspective view of a peripheral IV catheter to which the antimicrobial lubricant of the instant invention may be applied; and

FIG. 2 is a perspective view of the same peripheral IV catheter device but with the catheter tube or cannula having been removed from the needle.

### DETAILED DESCRIPTION OF THE INVENTION

The lubricious antimicrobial coating formed from the antimicrobial lubricant of the instant invention may be used to facilitate the insertion of a catheter (or catheter tube or cannula) in combination with a needle (or trocar) to a patient, and also possibly facilitate the relative movement of two portions of medical device, for example the movement of the needle relative to the catheter in a peripheral IV catheter device, such as the ProtectIV® product, or the Gripper® products sold by the Smiths Medical company. A description of the ProtectIV® product may be gleaned from U.S. Pat. No. 5,000,740, the disclosure of which is incorporated herein. One of the Gripper® products, with its associated infusion site or infuser, is disclosed in U.S. Pat. No. 7,549,976, the disclosure of which is incorporated by reference herein.

In both of the above-noted products, and other similar products, there is a catheter through which a needle is movably extended, so that the catheter, with the needle fully inserted in the catheter and the tip of the needle extending beyond the distal tip of the catheter, may be inserted into a patient. For the ProtectIV® product, the catheter is inserted directly into the patient, probably a vein of the patient. For the Gripper® product, the catheter is inserted into a port or reservoir that is implanted in the patient, so that medication may be conveyed to or fluid retrieved from the implanted port.

The lubricious antimicrobial coating on the outer diameter surface of the catheter facilitates the insertion of the catheter into the patient when the catheter makes contact interaction with the patient, as the lubricated outer diameter of the catheter is slippery and smooth. If either or both of the outer diameter surface of the needle and the inner diameter surface of the catheter is also coated with the inventive antimicrobial lubricant, the movement of those parts of the device relative to each other is likewise smoothly enhanced, for example when the needle is removed from the catheter after the combination catheter/needle has been properly inserted to the patient. Also, microorganism growth and possible fluid borne infections potentially carried in the interface between the needle and the catheter are prevented.

After the catheter is inserted into the patient, the antimicrobial property of the antimicrobial lubricant of the instant invention prevents or substantially eliminates, or at the very least stunts the growth of micro-organisms that otherwise may adhere to the catheter tubing and the needle, and the potential infection that may afflict the patient at the insertion or entry site and the areas of the patient adjacent to the site.

The antimicrobial lubricant of the instant invention is in a solution that contains a silicone oil lubricant having a solvent carrier, with the antimicrobial agent being mixed in the solution. The antimicrobial agent may be silver based and may include silver particles, carrier based ionic silver and other silver compounds. Silver based antimicrobial agents that may be used for the instant invention include the HyGenti™ 7000 and 8000 agents manufactured by the Ciba Company, now a subsidiary of the BASF Company. Another silver-based antimicrobial agent that may be used is the SilvaGard™ silver based antimicrobial agent by the AcryMed. Company. The SilvaGard™ antimicrobial agent, and its composition, is disclosed in US publications 2009/0035342, US 2007/003603 and U.S. Pat. No. 6,605,751. The respective disclosures of the '342 and 603 publications and the '751 patent are incorporated by reference herein. A non-silver based antimicrobial agent that may also be used is Triclosan sold under the trade name IRGASAN DP300 by the Ciba company.

For the antimicrobial lubricant of the instant invention, two preferred formulations of the silicone oil solution, which is used for lubricating medical devices, may be used. The lubricant in the silicone oil solution formulated per described below is polydialkylsiloxanes and preferably polydimethyl siloxane.

The first preferred silicone lubricant solution formulation uses approximately 2000 to 5000 grams, preferably between about 3000 to 3500 grams, of the volatile chemical solvent polydimethylcyclosiloxane composed mainly of decamethylcyclopentasiloxane (which is sold under the trade name ST-Cyclomethicone 5-NF by the Dow Corning Company) in combination with approximately 40 to 80 grams, preferably between 55 to 65 grams, of a non-volatile silicone compound poydimethylsiloxane (PDMS) having a viscosity of 12,500 centistokes (CSTKS). PDMS is sold by the Dow Corning company under the trade name 360 MEDICAL FLUID, 12,500 CST. The ST-Cyclomethicone 5-NF is combined with the 360 MEDICAL FLUID, 12,500 CST by a spinning or mixing process using any one of a number of conventional mixing machines known in the field. For this formulation, the spinning or mixing process of the two chemical components continues for a given time period, for example from a minimum of 10 minutes to about 30 minutes. In one particular practice of the formulation, an acceptable silicone oil solution was made by spinning or mixing about 3265 grams of St-Cyclomethicone 5NF with about 60 grams of the 360 MEDICAL FLUID, 12,500 CST for approximately 10 minutes.

The second preferred formulation of the silicone oil solution uses approximately 1500 to 3500 grams, preferably between about 2000 to 2500 grams, of volatile solvent methylsiloxane (sold under the trade name OS-10 by the Dow Corning company) in combination with approximately 150 to 400 grams, preferably between about 200 to 300 grams, of a not as volatile solvent solution that contains about 30% polydimethylsiloxane copolymers dispersed in xylene (sold under the trade name MED-4162 by the NuSil Technology Company). The OS-10 solvent and the MED-4162 solvent solution are combined by a spinning or mixing process using any one of a number of conventional mixing machines known in the field. For this formulation, the spinning or mixing process continues for a given time period, for example from a minimum of 10 minutes to about 30 minutes. In one particular practice of this formulation, an acceptable silicone oil solution was made by spinning or mixing about 2262 grams of OS-10 with about 250 grams of MED-4162 for approximately 10 minutes.

For both of the silicone lubricant solutions formulated per described above, the volatile component of the solution will flash off or evaporate after the solution is applied, for example by spraying onto a surface, such as for example the outer surface of a medical device, so that a film or layer of the non-volatile lubricant is left coated onto the surface. For example, the ST-Cyclomethicone 5-NF component of the lubricant solution of the first formulation will evaporate leaving a film of the non-volatile 360 MEDICAL FLUID, 12,500 CST component on the surface of the device as a lubricant therefor. For the lubricant solution processed from the second formulation, the volatile OS-10 component evaporates while the about 30% PDMS copolymers of the MED-4162 solvent solution remains as a lubricating coating on the surface of the device.

Continuing with the process of making the antimicrobial lubricant of the instant invention, an antimicrobial agent at approximately between 0.5 to 10% and preferably from 1.5 to 3% by weight based on the dry weight (the non-volatile component of the lubricant solution formulated per described above) is added to the silicone lubricant solution.

The antimicrobial agent added to the silicone lubricant solution may be one of the above-mentioned silver based agents, for example the HyGentic™ 7000, which is a glass carrier based silver antimicrobial, or the HyGentic™ 8000, which is a zeolite carrier based silver antimicrobial. The HyGentic™ 7000 may be represented as Ag-G (ionic silver on glass support), and the HyGentic™ 8000 may be represented as Ag-Z (ionic silver on zeolite support). Another antimicrobial agent that may be used to form the inventive antimicrobial lubricant but which is not silver based is Triclosan.

Adding one of the aforenoted antimicrobial agents to the lubricant solution effects an antimicrobial lubricant solution or formulation. The antimicrobial lubricant solution (in an appropriate container) then is placed in an ultrasonic machine, for example the Model 1510 Bransonic® Tabletop Ultrasonic cleaner manufactured by the Branson Ultrasonic Corporation. When energized, the ultrasonic machine uniformly mixes the antimicrobial agent in the lubricant solution by ultrasonic energy.

The mixed antimicrobial agent in the lubricant solution may resettle to the bottom of the lubricant solution after a prolong period of time if the lubricant solution is not used and remains still. To maintained the antimicrobial agent in the mixed state in the lubricant solution, the solution may be subjected to ultrasonic energy at given time intervals so as to prevent sedimentation of the silver based antimicrobial agent. The mixing of the antimicrobial agent in the lubricant solution may be done in either a batch or continuous process. Other methods that may be used to mix the antimicrobial agent in the silicone lubricant solution may include mechanical or electronic agitation methods, and the spinning and mixing process noted above. Heat or other chemicals that assist in keeping the silver particles mixed in the silicone lubricant solution may also be used.

The resulting antimicrobial lubricant solution may be applied to the medical device as a spray by using sprayers that include spray nozzles manufactured by the Precision Company located in Halfmoon, N.Y., and EFD Inc., a division of the Nordson Company located in East Providence, R.I. Instead of spraying the solution as a fine mist to coat the medical device, the medical device may be dipped into the antimicrobial lubricant solution, or the antimicrobial lubricant solution may be applied by other means such as an application tip or airbrush to the medical device.

An example of the use of the antimicrobial lubricant of the instant invention is illustrated with respect to the peripheral intravenous (IV) catheter device ProtectIV®, disclosed in the aforenoted U.S. Pat. No. 5,007,740.

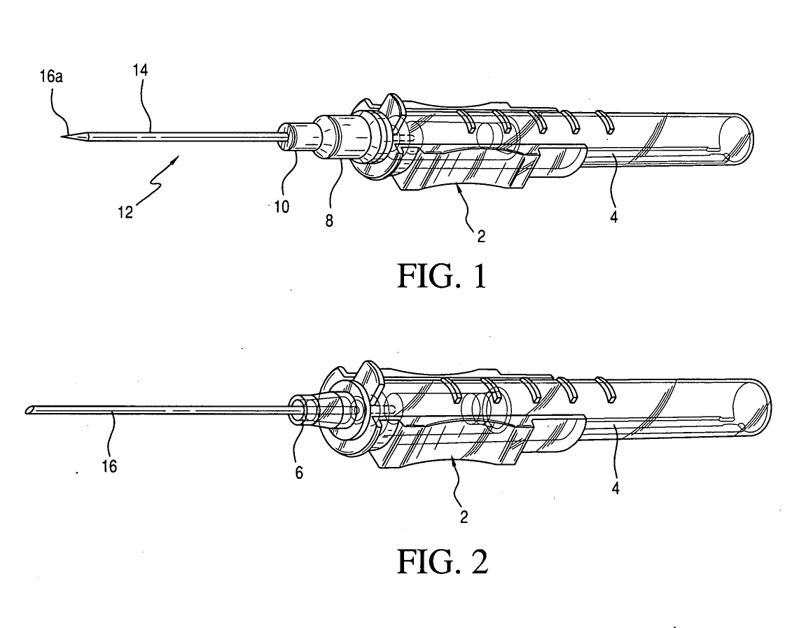

With reference to FIGS. 1 and 2, the peripheral IV catheter medical device has a needle housing 2 movable relative to a needle guard 4 that has a needle guard tip 6 to which the proximal end 8 of a needle hub or base 10 of a needle assembly 12 is fitted. Extending from base 10 is a catheter or cannula 14 through which a trocar or needle 16 extends and movable relative thereto. As shown in FIG. 1, the tip 16a of needle 16 extends out of the distal end of catheter 14 in the ready position for insertion to a patient. After insertion to the patient, needle 16 is withdrawn with the removal of needle assembly 2, so that only catheter 14 remains inserted to the patient.

The inventive antimicrobial lubricant is applied as a coating to the outer diameter surface along the entire length of catheter 14 to facilitate or ease the insertion of the combination needle 16 and catheter 14 to the patient. At the same time, given its antimicrobial attribute, the antimicrobial lubricant also acts as a deterrent to prevent or substantially eliminate the growth of microorganisms at the device, specifically the catheter, as it comes into contact with the patient during its entry into the patient. The antimicrobial agent in the antimicrobial lubricant may also prevent the growth of microorganisms within the body portion of the patient where the catheter has entered and is in contact with, and the area on the patient adjacent to where the device makes contact with the patient, as the antimicrobial agent from the catheter diffuses from the catheter out to its adjacent areas. Thus, those portions of the patient, and areas adjacent thereto, that come into contact with the device would be protected from the growth of microorganisms due to the antimicrobial attribute of the inventive antimicrobial lubricant.

For the instant invention, the antimicrobial coating is sprayed onto the outer diameter circumferential surface of the catheter 14 from a solution that contains the antimicrobial agent and the silicone lubricant, per described above. A second coat of the antimicrobial lubricant solution may be sprayed onto the outer diameter surface of the catheter after the volatile solvent carrier evaporates or flashes off from the first coating.

In addition to catheter 14, the needle hub 10 and its proximal portion 8 may also be sprayed with the antimicrobial lubricant so that the entire catheter assembly is covered with a lubricious antimicrobial coating of the instant invention. Furthermore, either the outer diameter of needle 16 or the inner diameter surface of catheter 14, or both of those surfaces, may be sprayed with the inventive antimicrobial lubricant to enhance the relative movement between the needle and the catheter. Also, the coating of the outer diameter needle and/or inner diameter catheter surfaces with the antimicrobial lubricant prevents the growth of micro-organisms and the potential carrying of fluid borne infections at the interface between the catheter and the needle.

Instead of spraying the antimicrobial lubricant to form a coating on the catheter and other medical devices such as the afore-mentioned Gripper® products, the catheter and the other medical devices may each be dipped into the antimicrobial lubricant solution of the instant invention.

In addition to the IV catheter as shown, other medical devices including infuser or infuser sites, implanted ports, catheter and needle devices for establishing a fluid path from the infusion site to the implanted port, and other catheters may also have their surfaces sprayed with the antimicrobial lubricant of the instant invention to attain both lubriciousness and antimicrobial protection for those devices.

In as much as the present invention is subject to many variations, modifications and changes in detail, it is intended that all matter described throughout this specification and shown in the accompanying drawings be interpreted as illustrative only and not in a limiting sense. Accordingly, it is intended that the invention be limited only by the spirit and scope of the hereto appended claims.

1. Medical device comprising a lubricious silver based antimicrobial coating that acts as a lubricant to facilitate the contact interaction of the device with a patient and a deterrent to prevent or substantially eliminate the growth of microorganisms on the device and at least those portions of the patient that the device comes into contact with to thereby prevent infection to the patient.

2. Medical device of claim 1, wherein the lubricious antimicrobial coating is formed on the medical device by spraying the medical device with a solution having a silver based antimicrobial agent mixed with a silicone lubricant.

3. Medical device of claim 2, wherein the solution comprises a silver antimicrobial agent with either zeolite carrier (Ag-Z) or glass carrier (Ag-G) mixed with the silicone lubricant, the solution having a volatile component and a non-volatile component.

4. Medical device of claim 1, wherein the lubricious antimicrobial coating is formed on the medical device by dipping the medical device into a silicone lubricant solution having a silver based antimicrobial agent mixed therein.

5. Medical device of claim 1, wherein the medical device includes any of the following: infusion sites and ports, inserters for the infusion sites and ports, cannula and needle devices, catheters including at least epidural and peripheral IV catheters.

6. Medical device of claim 1, wherein the medical device comprises a base, a catheter extending from the base and a needle extending through and movable relative to the catheter, and wherein at least the outer diameter surface of the catheter is coated by the lubricious antimicrobial coating.

7. Medical device of claim 6, wherein the outer surface of the base is also coated with the lubricious antimicrobial coating.

8. Medical device of claim 1, wherein the coating comprises a lubricant and an antimicrobial agent, and wherein the lubricant is a silicone based lubricant comprising polydialkylsiloxanes and preferably polydimethyl siloxane.

9. An antimicrobial lubricant comprising: a silver based antimicrobial agent combined with a silicone based lubricant, the antimicrobial lubricant adapted to coat a medical device for use with a patient, the antimicrobial lubricant acts as a lubricant to facilitate the contact interaction of the device with the patient, the antimicrobial lubricant further acting as a deterrent to prevent or substantially eliminate the growth of microorganisms on the device and at least those portions of the patient that the device comes into contact with to thereby prevent infection to the patient.

10. The antimicrobial lubricant of claim 9, wherein the silicone based lubricant comprises polydialkylsiloxanes and preferably polydimethyl siloxane.

11. The antimicrobial lubricant of claim 9, wherein the silver based antimicrobial agent comprises either Ag-Z (silver with zeolite carrier) or Ag-G (silver with glass carrier).

12. The antimicrobial lubricant of claim 9, wherein the antimicrobial agent is mixed with a silicone lubricant in a solution, the silver based antimicrobial agent having either zeolite carrier (Ag-Z) or glass carrier (Ag-G), the silicone lubricant having a volatile component and a non-volatile component, the volatile component flashing off after the medical device is coated with the antimicrobial lubricant so that the medical device is coated by the antimicrobial lubricant.

13. The antimicrobial lubricant of claim 9, wherein the antimicrobial lubricant is formed on the medical device by spraying the medical device with a solution having mixed therein the silver based antimicrobial agent and the silicone based lubricant.

14. The antimicrobial lubricant of claim 9, wherein the antimicrobial lubricant is formed on the medical device by dipping the device into a solution having mixed therein the silver based antimicrobial agent and the silicone based lubricant.

15. The antimicrobial lubricant of claim 9, wherein the medical device includes any of the following: infusion sites and ports, inserters for the infusion sites and ports, cannula and needle devices, and catheters including at least epidural and IV catheters.

16. The antimicrobial lubricant of claim 9, wherein the medical device comprises a catheter, a base wherefrom the catheter extends and a needle extending through and movable relative to the catheter, and wherein at least the outer diameter surface of the catheter is coated by the antimicrobial lubricant.

17. The antimicrobial lubricant of claim 16, wherein the outer surface of the base is also coated with the antimicrobial lubricant.

18. A catheter device comprising a needle having an outer diameter extending through and movable relative to a catheter having an inner diameter, the outer diameter surface of the needle in contact with the inner diameter surface of the catheter, the catheter having coated along its length at its outer diameter surface a lubricious antimicrobial coating that facilitates the insertion of the catheter and the needle to a patient and acts as a deterrent to prevent or substantially eliminate the growth of microorganisms at the device and at least those portions of the patient that the device comes into contact with to thereby prevent infection to the patient.

19. Catheter device of claim 18, wherein the lubricious antimicrobial coating comprises a silicone based lubricant and a silver based antimicrobial agent.

20. Catheter device of claim 19, wherein the silicone based lubricant comprises polydialkylsiloxanes and preferably polydimethyl siloxane.

21. Catheter device of claim 18, wherein the antimicrobial coating is formed on the outer diameter surface of the catheter by spraying thereon a solution having the silver based antimicrobial agent mixed with the silicone based lubricant, the silver based antimicrobial agent having either zeolite carrier (Ag-Z) or glass carrier (Ag-G), the silicone lubricant having a volatile component and a non-volatile component, the volatile component flashing off after the medical device is coated with the antimicrobial lubricant so that the medical device is coated by the antimicrobial lubricant.

22. Catheter device of claim 18, further comprising a base wherefrom the catheter extends, wherein the outer surface of the base is also coated with the lubricious antimicrobial coating.

23. An antimicrobial lubricant comprising an antimicrobial agent mixed with a silicone based lubricant in a solution, wherein the antimicrobial agent is Triclosan and the lubricant includes polydialkylsiloxanes preferably polydimethyl siloxane, the solution including a volatile solvent, wherein the Triclosan is mixed with the polydimethyl siloxane in the solution, the solution applied to the surface of a medical by spraying, whereby after the volatile solvent evaporates, the surface of the medical device is coated by a layer of the antimicrobial lubricant.

24. A method of making an antimicrobial lubricant comprising the steps of:
a) combining a solvent and a silicone base lubricant to formulate a lubricant solution;
b) adding a silver based antimicrobial agent in the lubricant solution;
c) subjecting the antimicrobial agent added lubricant solution to ultrasonic energy to mix the antimicrobial agent with the silicone based lubricant to form an antimicrobial lubricant solution; and
d) evaporating the solvent from the antimicrobial lubricant solution to obtain the antimicrobial lubricant.

25. The method of claim 24, wherein the silicone based lubricant comprises polydialkylsiloxanes and preferably polydimethyl siloxane.

26. The method of claim 24, wherein the antimicrobial agent comprises either silver with zeolite carrier (Ag-Z) or silver with glass carrier (Ag-G).

27. The method of claim 24, comprising the step of spraying the antimicrobial lubricant solution on a medical device before step (d) so that the antimicrobial lubricant is formed as a lubricious antimicrobial coating on the medical device.
